(19) United States
(12) Patent Application Publication
(10) Pub. No.: US 2025/0255888 A1
(43) Pub. Date: Aug. 14, 2025

(54) Use of Baby Aspirin with Diphenhydramine or other Sleep Aids

(71) Applicant: Joseph O. Udeaja, Chattanooga, TN (US)

(72) Inventor: Joseph O. Udeaja, Chattanooga, TN (US)

(21) Appl. No.: 18/436,320

(22) Filed: Feb. 8, 2024

(51) Int. Cl. A61K 31/616; A61K 31/138

(52) U.S. Cl. CPC A61K 31/616; A61K 31/138

(57) ABSTRACT

Aspirin (2-Acetoxybenzoic Acid) and Benadryl (diphenhydramine HCL) are widely known medications used for different medical problems. Each of these medications has been studied extensively however, a review did not identify any histologic investigation into combining the two entities together for any intended use such as for the management of |Benadryl-induced CNS-related adverse effects specifically, poor sleep quality, lingering drowsiness or difficulty concentrating, especially when enough sleep (about 7 hours) was not obtained. In these situations, patients often try several agents and practices to counteract Benadryl-induced aftereffects often with limited success.

The patent applicant had experience with occasionally taking Benadryl tablet or liquid for sleep however, each time in the morning, he would experience some of the aftereffects of Benadryl such as poor sleep quality, lingering drowsiness or difficulty concentrating. These effects were worse each time he did not get at least, seven hours of sleep. One night, he decided to take one tablet of Aspirin 81 mg (“baby aspirin”) with Benadryl and noticed that he not only slept well but did not experience any of the above aftereffects in the morning. He tried taking the same combination few more times and each time, did not experience any of the aftereffect, even when he did not get up to seven hours of sleep. He further recommended same to two of his close friends who had complained of same aftereffects of Benadryl. Each reported similar result: no significant aftereffects.

When aspirin was used with other sleep aids that contain diphenhydramine as an active ingredient, similar result was obtained.

Thus, this Patent Application is for a product resulting from the combination of Aspirin in all forms: tablet, liquid, capsule, and, in all dosage strengths.

And,

Benadryl (diphenhydramine HCL) in all forms: tablet, capsule, liquid, and, in all dosage strengths.

1. A product resulting from combination of Aspirin (2-Acetoxybenzoic Acid) and Benadryl (diphenhydramine HCL).

2. A product resulting from combination of Aspirin (2-Acetoxybenzoic Acid) and any product containing diphenhydramine HCL as the active ingredient.
